(19) United States
(12) Patent Application Publication
(10) Pub. No.: US 2014/0175189 A1
(43) Pub. Date: Jun. 26, 2014

(54) Refill for a Liquid

(71) Applicant: Helen Stephenson, Hull (GB)

(72) Inventor: Helen Stephenson, Hull (GB)

(73) Assignee: Reckitt & Colman (Overseas) Limited, Slough, Berkshire (GB)

(21) Appl. No.: 14/127,642

(22) PCT Filed: Jun. 22, 2012
(86) PCT No.: PCT/GB2012/051453

§ 371 Date: Feb. 21, 2014

(30) Foreign Application Priority Data
Jun. 24, 2011 (GB) 1110689.5

(51) Int. Cl. A61L 9/12

(52) U.S. Cl. CPC A61L 9/127
USPC 239/49

(57) ABSTRACT

A refill for a liquid wherein the refill comprises: a housing; at least one reservoir in the housing for holding the liquid; at least one aperture in the housing sealed by at least one valve wherein said at least one valve defines a liquid pathway from the reservoir to the exterior of the refill, wherein at least one of said valve(s) is configured to be automatically resealable when not being held open; and at least one wick housed entirely within the housing.

### FIELD OF THE INVENTION

The present invention relates to devices and methods for improved airborne delivery of liquids containing one or more active materials wherein the active material comprises at least one of: a fragrance; an insecticide; a fungicide; a pesticide; a sanitising material; and/or a pharmaceutical.

### BACKGROUND

Liquids, and commonly volatile liquids, containing one or more active materials wherein the active material comprises at least one of: a fragrance; an insecticide; a fungicide; a pesticide; a sanitising material; and/or a pharmaceutical are delivered within the domestic environment via a variety of mechanisms. Devices are available with heaters disposed therein to increase the rate of emanation from a surface saturated with the liquid, such a surface could be a wick saturated with a fragranced liquid and the heater is located adjacent the wick surface and nearby a chimney to heat the liquid on the wick surface and cause it to more readily evaporate and disseminate into the surrounding environment through the chimney.

Common wick and heater emanation systems typically comprise a refill of liquid and an emanation device containing a heater. In these common systems the refill consists of a bottle of liquid, typically a volatile liquid, wherein the bottle is sealed with a wick holder that contains a central aperture which grips a wick that extends from the base of bottle and through the wick holder to extend a short distance above the holder; the part of the wick which extends above the holder is the exposed part of the wick from where all emanation of the liquid takes place. When the refill is connected to the emanation device, the wick extends into a chimney of the device and the device has a heater located adjacent to the chimney to heat the wick directly or indirectly via the chimney to cause evaporation of the liquid from the exposed part of the wick. In use evaporated liquid travels up the chimney and out into the surround environment, the liquid in the exposed part of the wick is replenished due to the wicking/capillary action of the wick drawing up more liquid from the bottle.

One drawback with known refills containing a wick is that the efficiency of a particular wick material depends on the nature of the liquid, or component liquids within the liquid. Most available wick materials, such as those mentioned above, have a limited porosity and liquid transfer rate/evaporation rate due, at least in part, to fractionation and/or blocking/clogging of the wick. Fractionation over time will change the character and/or intensity of the active and will slow evaporation, this is particularly noticeable for fragrances wherein common wicks cause the ‘high notes’ of a fragrance to be evaporated when the wick is first exposed to the fragranced liquid, and the ‘low notes’ are evaporated thereafter which affects the user's experience.

The present invention is concerned with providing an improved refill and emanation device for a wick and heater emanation system that addresses many of the drawbacks associated with such systems.

### SUMMARY OF INVENTION

According a first aspect of the present invention there is provided therefore a refill for a liquid wherein the refill comprises:

a housing;

at least one reservoir in the housing for holding the liquid;

at least one aperture in the housing sealed by at least one valve wherein said at least one valve defines a liquid pathway from the reservoir to the exterior of the refill, wherein at least one of said valve(s) is configured to be automatically resealable when not being held open;

and at least one wick housed entirely within the housing.

According a second aspect of the present invention there is provided therefore a refill of liquid containing one or more active materials wherein the refill comprises:

a housing;

at least one reservoir in the housing holding the liquid;

at least one aperture in the housing sealed by at least one valve wherein said at least one valve defines a liquid pathway from the reservoir to the exterior of the refill, wherein at least one of said valve(s) is configured to be automatically resealable when not being held open;

and at least one wick housed entirely within the housing.

Although a plurality of apertures may be provided, preferably the housing has a single aperture. The aperture is preferably located in an upper wall of the housing. It is to be understood that reference to an “upper wall” is made relative to the other walls of the housing purely for the purpose of spatially describing the refill and, unless otherwise stated, is not to be understood as imparting any restrictive orientation on the refill itself.

The aperture(s) is preferably located in a substantially central position of said upper wall or located in a position removed from the periphery of the upper wall.

The upper wall of the housing preferably faces a lower wall and is connected thereto by one or more side walls. When the valve(s) in the aperture(s) is held in an open position, an elongate column of space within the interior of the housing extending from the valve(s) to the lower wall of the housing is defined, hereinafter referred to as the elongate column.

The at least one wick preferably substantially surrounds the elongate column. Most preferably one wick is provided and said one wick is hollow in order to surround the elongate column. Alternatively the said one wick may be C-shaped or the like to partially or substantially surround the elongate column. As a further alternative a plurality of wicks may be provided which are spaced apart from each other to partially or substantially surround the elongate column.

The at least one wick may be fixed within the housing to the upper wall or adjacent thereto and extends therefrom toward the lower wall to terminate adjacent thereto or terminate by contacting the lower wall. Alternatively the at least one wick may be fixed within the housing to the lower wall or adjacent thereto and extends therefrom toward the upper wall to terminate adjacent thereto or terminate by contacting the upper wall. As a further alternative, the at least one wick may be fixed at one end within the housing to the upper wall or adjacent thereto and is fixed at its other end to the lower wall or adjacent thereto. Such arrangements permit the wick(s) to be well placed to contact liquid held within the reservoir almost regardless of the orientation of the refill.

Although the refill may have more than one reservoir, and it is preferable that when there is more than one reservoir more than one wick is also present wherein no wick has access to more than one reservoir. The most preferred arrangement however comprises the refill having a single reservoir and preferably the boundaries of the single reservoir are provided by inner surfaces of the housing walls.

The at least one valve may be provided by a single automatically resealable valve. The single valve may be provided in the form of a self-sealing liquid-tight valve, such as a silicone valve or the like. Alternatively the single valve may be provided in the form of a movable sealing closure means that is biased towards a closed position by a biasing means, in this arrangement a sealing means such as an O-ring or the like may also be located around the movable sealing closure means to ensure a liquid-tight seal when said closure means is in a closed position and/or the sealing means such as an O-ring may be located in the periphery of the aperture to add a sealing function against any means that are not a part of the refill used to open the closure means.

In a preferred arrangement however the, or each, aperture is provided with at least two valves. When at least two valves are provided a downstream valve (i.e. the valve closest to the reservoir) and an upstream valve is provided. The downstream valve is preferably provided by a self-sealing liquid-tight valve, such as a rubber, rubberised, silicone slit or cross valve or the like, wherein the valve tends toward a closed position when not being held open, or provided by a sealing means biased into a closed position by a deformable biasing means such as a spring means or the like. The upstream valve is preferably provided as an annular ring which is preferably an open ring that is not able to prevent fluid flow therethrough. Preferably the annular ring is supported on a flexible annular skirt. The opening in the annular ring is preferably provided with a diameter of between 0.1-20.0 mm, and more preferably with a diameter of between 3.0-15.0 mm, and even more preferably with a diameter of between 4.0-10.0 mm, and most preferably with a diameter of between 5.0-7.0 mm. Whilst the annular ring is preferably provided in a circular shape other shapes may be permissible providing the shape is capable of performing the sealing function required of it, such shapes include substantially circular shapes, oval shapes, diamond shapes and such like.

The downstream valve is preferably operable to prevent any fluid within the refill from escaping until the downstream valve is opened and the upstream valve is operable to sealingly engage with liquid extraction means that are part of the emanation device which enter the refill to open a liquid pathway from the refill into the device. In this arrangement the downstream and upstream valves cooperate in that the downstream valve does not need to be optimised to seal against the liquid extraction means that open the valve as the upstream valve can be configured to undertake that task and, vice versa, the upstream valve need not be optimised to form a liquid-tight seal as the downstream seal can be optimised for that task. Furthermore, the open appearance of the upstream valve provides a user with a visual cue to aim either the liquid extraction means from the emanation device or use as a guide when loading the refill onto said means.

In one preferred embodiment the downstream valve and the upstream valve are formed as separate pieces that are held adjacent but spaced apart from each other. In a most preferred embodiment however the downstream valve and the upstream valve are formed as a one piece component.

In a most preferred embodiment the refill is provided with a single aperture closed by a valve system comprising two valves wherein one of said valves is configured to be automatically resealable when not being held open and the other of said valves is provided in the form of an annular ring valve.

Advantageously the valve system(s) of the present invention may ameliorate, and for some types of liquid substantially completely remove, the damaging effects of fractionation as all the liquid and any naturally volatilised quantity thereof are retained within the refill until extraction is required.

Said at least one valve could be adjustable to affect the flow rate of the liquid therethrough. The adjustability may be facilitated manually by a user and/or due to an automated function of a device to which the refill is connected and said automated function may be controlled automatically by the device or may be in response to a user input into the device. Adjustability may be particularly useful when the device is configured to emanate the liquid passively and/or extract the liquid passively such as by gravity feed.

The housing may be made of any material suitable for retaining a liquid containing one or more active materials, such suitable materials include glass, certain plastics materials and the like. The housing is preferably made from a material that is transparent or at least translucent or is provided with a section thereof adjacent the reservoir(s) that is substantially transparent or translucent to permit a user to easily determine whether the refill contains any liquid and/or the level of said liquid.

The wick(s) may be made of any material that is capable of wicking a liquid, such suitable materials include plastics materials such as cintered polyethylene, cellulose, woods such as balsa or bamboo, reeds such as rattan.

The refill may be provided with one or more vent holes in the housing, said vent holes being provided in the form of a one-way valve that is in communication with the reservoir but does not facilitate a liquid pathway therefrom rather it solely permits air to enter the reservoir from the outside of the refill to prevent or reduce any build up of negative air pressure within the refill as liquid is removed therefrom. The vent hole(s) may be covered with a gas permeable membrane. Preferably however, the vent hole(s) is not covered and is sized such that the liquid may not escape therefrom or that the rate of escape would be sufficiently low as to not be relevant for the safe operation of the refill with a device configured to operate with the refill to emanate liquid obtained therefrom.

The refill may further comprise one or more diptubes therein.

Any of the features described herein may be combined with any of the above aspects in any combination.

### BRIEF DESCRIPTION OF THE DRAWINGS

Embodiments of the invention will now be described, by way of example only, with reference to the following drawings in which:

FIG. 1 shows a perspective view of the refill;

FIG. 2 shows a perspective view of an extraction limb of an emanation device;

FIG. 3 shows a sectional view of the refill valve;

FIG. 4 shows a sectional view of the refill with a mechanism for a gravity-feed extraction emanation device;

FIG. 5 shows a sectional view of the refill with a mechanism for a heated extraction emanation device; and

FIG. 6 shows a sectional view of the refill with a mechanism for a blown air extraction emanation device.

### DESCRIPTION OF AN EMBODIMENT

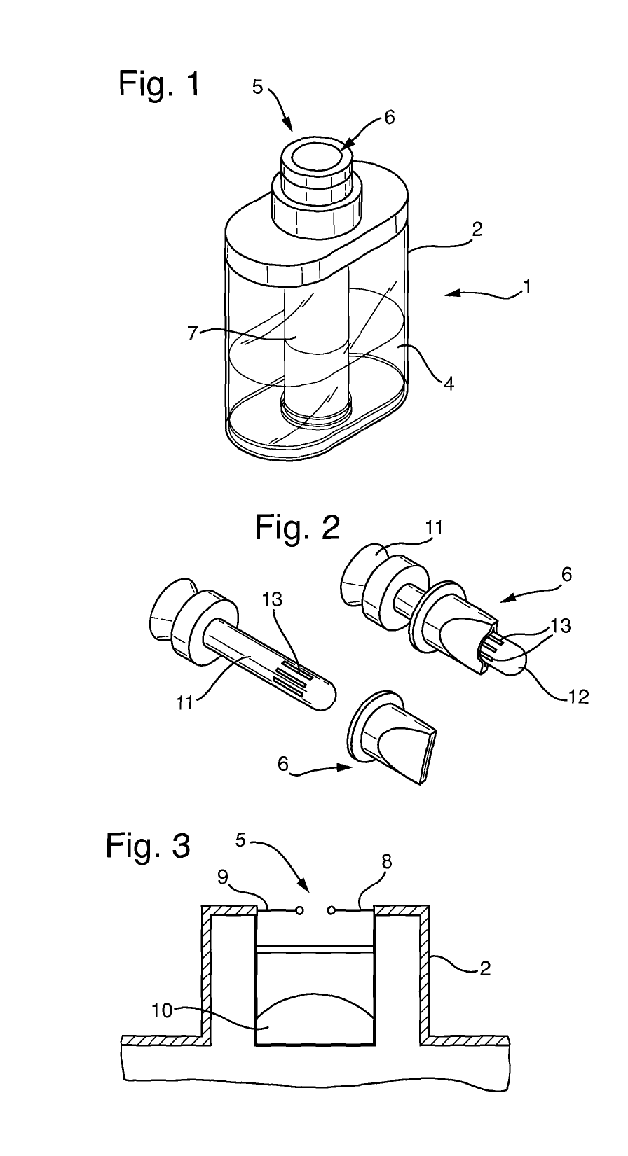

FIG. 1 shows a preferred embodiment of a refill 1 according to the present invention. The refill 1 comprises a housing 2 that surrounds and contains a reservoir 3 of liquid 4 therein. Access to the interior of the refill 1 and the reservoir 3 is provided via aperture 5 which is sealed by a valve 6. Housed entirely within the housing 2 of the refill 1 is a wick 7. The wick 7 is shown in FIG. 1 as a having a substantially cylindrical shape that extends from adjacent the valve 5 to contact a base of the reservoir 3.

Although not shown in FIG. 1, when the valve 6 is held in an open position, a notional elongate column of space within the interior of the housing extending from the perimeter of the valve opening to the lower wall of the housing is defined, hereinafter referred to as the elongate column. The wick 7 has a cylindrical shape that is hollow to substantially surrounds the elongate column.

FIG. 3 shows the valve 6 is greater detail. The valve 6 is an automatically resealable valve system comprising an open annular ring valve 8 suspended by a flexible annular skirt 9 which forms the upstream valve and the valve system further comprises a downstream valve in the form of a rubberised slit valve 10. The downstream slit valve 10 provides a fluid tight closure to prevent the liquid in the reservoir from escaping.

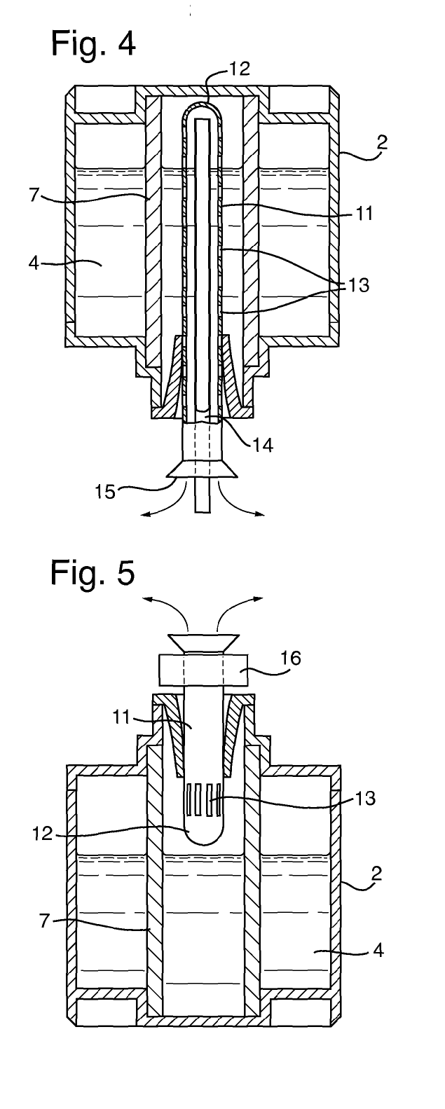

FIG. 4 shows how an emanation device can interact with the valve system of the refill 1. A hollow extraction limb 11 having a rounded end section 12 may be provided operatively connected to the emanation device. The limb 11 is provided with a series of perforations 13 which provide access to the hollow interior of the limb 11. Preferably the limb 8 is sized to have a diameter that is slightly wider than the diameter of the ring valve 8 such that, in use, when the rounded end 12 is pushed through the ring valve 8 a liquid-tight connection is made between the limb 11 and the ring valve 8. On travelling further into the valve 6 the rounded end will meet the slit valve 10 and force it open. Since the limb 11 and the ring valve 8 have formed a liquid-tight connection any liquid that is not able to flow past the slit valve 10 will be prevented from leaking out of the refill 1. The limb 11 will be pushed into the valve 5 such that the perforations 13 also pass the slit valve 10 to be disposed within the interior of the refill 1.

A mechanism that is suitable for a gravity-fed emanation device is shown in FIG. 4. In this arrangement the refill 1 is loaded into the device in an inverted orientation such that the extraction limb 11 is generally upstanding and the valve 5 of the refill is generally the lowest part thereof. In this arrangement it can be seen that the extraction limb comprises a hollow limb with perforations 13 and further comprising a central hollow core 14 without perforations but with open ends. To extract the liquid 4 from the refill 1 in this arrangement, the refill 1 is loaded on to the extraction limb 11 to form a liquid-tight seal between the limb 11 and the ring valve 8. Liquid is permitted to enter the hollow interior of the limb 11 via the perforations 13 but the liquid is not present in a sufficient volume to enter the open end of the central hollow core 14. When the device is activated the liquid may flow in a downward direction powered by gravity and toward an open end 15 of the limb 11 and into the device. The central hollow core 14 may be open to the atmosphere or connected to an air pump in order to return air to the reservoir 3 of the refill 1 to prevent negative pressure from developing within the refill 1 and retarding or choking the flow of liquid 4 into the device.

A mechanism that is suitable for a heated emanation device is shown in FIG. 5. In this arrangement the refill 1 is loaded into the device in an upright orientation such that the valve 5 is generally the uppermost part of the refill 1. Again the refill 1 is loaded on to the extraction limb 11 to form a liquid-tight seal between the limb 11 and the ring valve 8, and preferably the limb enters the refill such that the perforations 13 are below the slit valve but also so that the rounded end 12 is not in contact with the liquid 4. The extraction limb 11 is provided with a heater 16 connected thereto. The heater 16 is configured to heat the extraction limb 11, the limb 11 being made from a suitably heat-conductive material, in order to transmit heat to the interior of the refill 1. The heater 16 is configured to transmit enough heat to the interior of the refill 1 to volatilise the liquid held in the wick 7, the resulting volatilised liquid may then enter the perforations 13 and travel upwardly into the device and out into the surrounding environment.

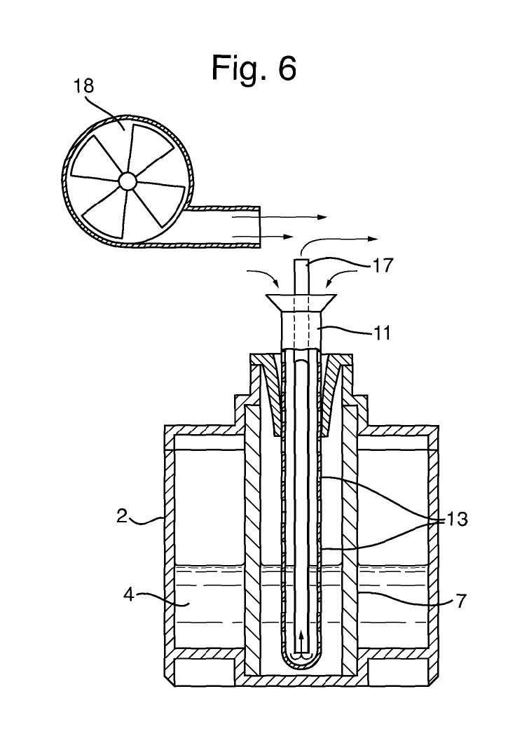

A mechanism that is suitable for a heated emanation device is shown in FIG. 6. In this arrangement the refill 1 is loaded into the device in an upright orientation such that the valve 5 is generally the uppermost part of the refill 1. The refill 1 is loaded on to the extraction limb 11 to form a liquid-tight seal between the limb 11 and the ring valve 8, and preferably the limb enters the refill such that the perforations 13 are below the slit valve and also below the liquid level, to this end the perforations 13 may be adjacent to the rounded end 12. The limb 11 may also be provided with a central hollow liquid conduit 17 that is open at both ends. The upper end of the conduit 17 may be adjacent a an air pump 18 that is configured to blow air across the top of the conduit 17 to cause liquid to travel up the conduit by both capillary action and the Venturi effect and be blown into the surrounding environment. In this arrangement air would be permitted to be returned into the reservoir 3 to prevent negative pressure from developing within the refill 1 and retarding or choking the flow of liquid 4 into the device by air travelling within the hollow limb 11 between an inner surface of the limb and an outer surface of the conduit and passing into the reservoir through the perforations.

Although not shown, the end of the housing 1 containing the valve 6 may be substantially square in shape and guide means (not shown) in a chassis (not shown) of each emanation device) could cooperate with grooves in the refill housing (not shown) to ensure that the refill could only be engaged with the chassis if the limb 11 is correctly aligned to open valve 6. Although not illustrated, the end of the refill housing containing the valve 6 could be shaped such that the loading thereof into the chassis was only possible via a single orientation of that end of the refill.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A refill for a liquid wherein the refill comprises:
a housing;
at least one reservoir in the housing for holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
and at least one wick housed entirely within the housing.

2. A refill of liquid containing one or more active materials wherein the refill comprises:
a housing;
at least one reservoir in the housing holding the liquid;
at least one aperture in the housing sealed by at least one valve wherein said at least one valve defines a liquid pathway from the reservoir to the exterior of the refill, wherein at least one of said valve(s) is configured to be automatically resealable when not being held open;
and at least one wick housed entirely within the housing.

3. A refill according to claim 1, wherein the housing has a single aperture.

4. A refill according to claim 1, wherein the at least one wick substantially surrounds an elongate column defined by an elongate column of space within the interior of the housing extending from the valve(s) to a lower wall of the housing.

5. A refill according to claim 1, wherein one wick is provided and said one wick is hollow and surrounds the elongate column.

6. A refill according to claim 1, wherein one wick is provided and said one wick is C-shaped or the like to partially or substantially surround the elongate column.

7. A refill according to claim 1, wherein a plurality of wicks are provided which are spaced apart from each other to partially or substantially surround the elongate column.

8. A refill according to claim 1, wherein the at least one valve is provided by a single automatically resealable valve.

9. A refill according to claim 8, wherein the valve is provided in the form of a self-sealing liquid-tight valve.

10. A refill according to claim 8, wherein the valve is provided in the form of a movable sealing closure means that is biased towards a closed position by a biasing means.

11. A refill according to claim 1, wherein the, or each, aperture is provided with at least two valves comprising a downstream valve and an upstream valve.

12. A refill according to claim 11, wherein the downstream valve is provided by a self-sealing liquid-tight valve, wherein the downstream valve tends toward a closed position when not being held open, or is provided by a sealing means biased into a closed position by a deformable biasing means such as a spring means or the like.

13. A refill according to claim 12, wherein the upstream valve is provided as an annular ring which is an open ring that is not able to prevent fluid flow therethrough.

14. A refill according to claim 13, wherein the annular ring is supported on a flexible annular skirt.

15. A refill according to claim 2, wherein the housing has a single aperture.

16. A refill according to claim 2, wherein the at least one wick substantially surrounds an elongate column defined by an elongate column of space within the interior of the housing extending from the valve(s) to a lower wall of the housing.

17. A refill according to claim 2, wherein one wick is provided and said one wick is hollow and surrounds the elongate column.

18. A refill according to claim 2, wherein the at least one valve is provided by a single automatically resealable valve.

19. A refill according to claim 18, wherein the valve is provided in the form of a self-sealing liquid-tight valve.

20. A refill according to claim 18, wherein the valve is provided in the form of a movable sealing closure means that is biased towards a closed position by a biasing means.
